(19) United States
(12) Patent Application Publication
(10) Pub. No.: US 2020/0281963 A1
(43) Pub. Date: Sep. 10, 2020

(54) HYDROGEN MOLECULE-CONTAINING PROPHYLACTIC OR THERAPEUTIC AGENT FOR OXIDATIVE DAMAGE IN INTRAOCULAR SURGERIES

(71) Applicants: Hiroshi TAKAHASHI, Tokyo (JP); Ikuro OSAWA, Tokyo (JP); Tsutomu IGARASHI, Tokyo (JP)

(72) Inventors: Hiroshi TAKAHASHI, Tokyo (JP); Ikuro OSAWA, Tokyo (JP); Tsutomu IGARASHI, Tokyo (JP)

(21) Appl. No.: 16/071,310

(22) PCT Filed: Jan. 20, 2017
(86) PCT No.: PCT/JP2017/002838

§ 371 Date: Jul. 19, 2018

(30) Foreign Application Priority Data
Jan. 21, 2016 (JP) 2016-009784

(51) Int. Cl. A61K 33/00

(52) U.S. Cl. CPC A61K 33/00; A61K 9/0048

(57) ABSTRACT

The present invention relates to a hydrogen molecule-containing prophylactic or therapeutic agent for oxidative damage for use in intraocular surgery, which is intended to provide a prophylactic or therapeutic agent for oxidative damage, which is administered when phacoemulsification cataract surgery is performed.

### TECHNICAL FIELD

The present invention relates to a prophylactic or therapeutic agent for oxidative damage generated in intraocular surgery. The present invention particularly relates to a prophylactic or therapeutic agent for oxidative damage, which is administered to suppress corneal disorder generated as a result of phacoemulsification cataract surgery.

### BACKGROUND ART

Since surgical operation itself is an invasive action on tissues, it eventually causes oxidative stress to the tissues. In particular, during intraocular surgery under operating microscope, the eyeball is illuminated with a strong light and mechanically compressed. As such, ocular tissues are exposed to oxidative stress caused by photodamage or transient focal ischemia-reperfusion injury. Since the impairment of cornea or retinal tissues caused by such oxidative stress would be a factor of deteriorating prognosis, it has been desired to develop an appropriate therapeutic method for oxidative stress.

Cataract means a state, in which a crystalline lens is clouded. The number of patients suffering from cataract tends to be increased together with aging. According to the statistics of the Japanese Ministry of Health, Labour and Welfare, the total number of Japanese patients with cataract surgery is approximately 1,000,000. It is necessary to remove a clouded crystalline lens from the eyeball of a cataract patient, and then to make the pupillary zone transparent. Phacoemulsification cataract surgery is the most popular intraocular surgery. Phacoemulsification is, at present, a main cataract surgery for adults and aged people, and this is a surgical method comprising circumferentially excising the anterior capsule of lens, then disintegrating the nucleus of the lens by ultrasound, and then aspirating the emulsified nucleus and cortex. Ultrasound exhibits a mechanical action serving as destructive energy on living bodies. A main reason for such a mechanical action is cavitation. A main reason for such a mechanical action is cavitation, which is a phenomenon whereby air dissolved in a liquid is changed to fine air bubbles by ultrasonic wave. Using such ultrasound, the nucleus is disintegrated. However, ultrasonic oscillation occurs in the anterior chamber sandwiched between the iris and the cornea, disorder is given to corneal endothelial cells. It is suggested that this disorder be caused by the indirect action of oxidative stress generated. As a result of the aforementioned disorder, if the cataract is transferred to bullous keratopathy, transplantation of a cornea obtained from a donor is necessary for the treatment of the bullous keratopathy. Bullous keratopathy is caused by cataract surgery, occurs at a rate of approximately one out of 1000 cases. In Japan, approximately 1000 cataract patients who have undergone cataract surgery have experienced transition to bullous keratopathy, and this has been considered to be a serious problem in clinical sites.

Regarding intraocular surgery, it has been known that oxidative stress is also generated in vitrectomy, and that this oxidative stress affects the prognosis. Vitrectomy, sometimes called vitreous surgery, is an operation to excise clouded vitreous body and to replace it with artificial aqueous humor, or gas bubble, silicon oil, etc. Conventionally, it has been reported that oxidative damage is generated in retinal photoreceptor cells as a result of high level of light exposure during intraocular surgery. Also in vitreous surgery, it has been problematic that light exposure caused by intraocular illumination used during the surgery induces cytotoxicity attended with oxidative stress, such as an increase in lipid peroxide. Moreover, it has been reported that the concentration of ascorbic acid in the remaining vitreous body is decreased even by simple excision of anterior vitreous body, and thus, it has been suggested that ascorbic acid, which has been diffused from ciliary epithelial cells into vitreous body, be likely to be consumed by oxidative stress generated as a result of excision of the vitreous body. Furthermore, it has been considered that, even in retinal disorder attended with air perfusion or retinal disorder caused by a staining solution such as indocyanine green, free radicals, as well as mechanical factors, are largely involved. It has been suggested that the operations of vitreous surgery themselves be likely to become oxidative stress to retinal cells.

An attempt has been made to suppress oxidative damage caused by intraocular surgery (see Patent Literature 1). Upon performing phacoemulsification cataract surgery, a viscoelastic material (sodium hyaluronate) is injected into the anterior chamber. However, the effect of the viscoelastic material to suppress oxidative damage is limited. The effects of vitamin C have been experimentally demonstrated, but at the same time, it may cause side effects. Thus, it has not yet been put to practical use. Regarding vitreous surgery, the effectiveness of a radical scavenger formulation has been reported. However, such radical scavenger formulations have limitation in terms of the ability to reach tissues with disorder, depending on the administration method, the molecular weight of a drug, etc. Also, the possibility of the side effects cannot be denied. Hence, under the current circumstances, there are no appropriate means for suppressing oxidative damage in intraocular surgery.

### CITATION LIST

### Patent Literature

### Patent Literature 1: JP Patent Publication (Kohyo) No. 2003-507419 A

### SUMMARY OF INVENTION

It is an object of the present invention to provide a hydrogen molecule-containing prophylactic or therapeutic agent for oxidative damage generated in intraocular surgery, and particularly, a hydrogen molecule-containing prophylactic or therapeutic agent for oxidative damage, which is administered upon performing phacoemulsification cataract surgery.

The present inventors have found that, in a corneal disorder model experiment involving intracameral ultrasonic vibration, using rabbits, corneal opacity that is an indicator for oxidative damage is significantly reduced by perfusing the anterior chamber with a hydrogen-containing irrigating solution of a hydrogen molecule-containing prophylactic or therapeutic agent for oxidative damage. This can significantly improve prophylactic or therapeutic effects, upon performing intraocular surgeries such as phacoemulsification cataract surgery.

Specifically, the present invention is the following.

[1] A prophylactic or therapeutic agent for oxidative damage, comprising hydrogen molecules, wherein the prophylactic or therapeutic agent is for use in intraocular surgery.
[2] The prophylactic or therapeutic agent for oxidative damage for use in intraocular surgery according to the above [1], wherein the prophylactic or therapeutic agent comprises hydrogen molecules at a concentration of 0.4 mM or higher.
[3] The prophylactic or therapeutic agent for oxidative damage for use in intraocular surgery according to the above [1] or [2], wherein the prophylactic or therapeutic agent is an irrigating solution.
[4] The prophylactic or therapeutic agent for oxidative damage for use in intraocular surgery according to any one of the above [1] to [3], which is administered during phacoemulsification cataract surgery.
[5] The prophylactic or therapeutic agent for oxidative damage for use in intraocular surgery according to the above [4], which is continuously administered to eyes via perfusion administration during intraocular surgery.
[6] The prophylactic or therapeutic agent for oxidative damage for use in intraocular surgery according to the above [4], which is used in combination with injection of a viscoelastic material.

Oxidative damage caused by surgical invasion, light injury, transient ischemia-reperfusion injury and the like can be prevented by performing intraocular perfusion with the hydrogen molecule-containing prophylactic or therapeutic agent for oxidative damage for use in intraocular surgery of the present invention, during intraocular surgeries such as phacoemulsification cataract surgery, and also, a good prognosis can be maintained, and prophylactic or therapeutic effects can be enhanced.

The present description includes the contents as disclosed in Japanese Patent Application No. 2016-009784, which is a priority document of the present application.

### BRIEF DESCRIPTION OF DRAWINGS

FIG. 1 is a view showing a change in the hydrogen concentration of a prophylactic or therapeutic agent for oxidative damage for use in intraocular surgery, which has been indwelled in an acrylic box filled with hydrogen gas for 24 hours and has been then removed from the box.

FIG. 2 is a view showing a change in the hydrogen concentration of a prophylactic or therapeutic agent for oxidative damage for use in intraocular surgery, which has been indwelled in an acrylic box filled with hydrogen gas for 1 week and has been then removed from the box.

FIG. 3 is a view showing opacity (the portion indicated with the arrow in the view) caused by corneal edema during the use of a normal irrigating solution.

FIG. 4 is a view showing the state of cornea (no opacity is seen) in the case of using a hydrogen-containing irrigating solution.

FIG. 5 is a view showing opacity (clouded) caused by corneal edema during the use of a normal irrigating solution.

FIG. 6 is a view showing the state of cornea (highly transparent) in the case of using a hydrogen-containing irrigating solution.

FIG. 7 is a view showing significant suppression of the turbidity of the cornea that has been increased by ultrasound, using a hydrogen-containing irrigating solution.

FIG. 8 is a view showing significant suppression of HO-1 induced by oxidative stress, using a hydrogen-containing irrigating solution.

FIG. 9 is a view showing 4-HNE-positive corneal endothelial cells, which have appeared as a result of an ultrasonic treatment.

FIG. 10 is a view showing 4-HNE-positive corneal endothelial cells, which have appeared as a result of an ultrasonic treatment upon the use of a hydrogen-containing irrigating solution.

FIG. 11 is a view showing significant suppression of the number of 4-HNE-positive corneal endothelial cells, which have appeared by an ultrasonic treatment, using a hydrogen-containing irrigating solution.

FIG. 12 is a view showing 8-OHdG-positive corneal endothelial cells, which have appeared as a result of an ultrasonic treatment.

FIG. 13 is a view showing 8-OHdG-positive corneal endothelial cells, which have appeared as a result of an ultrasonic treatment upon the use of a hydrogen-containing irrigating solution.

FIG. 14 is a view showing significant suppression of the number of 8-OHdG-positive corneal endothelial cells, which have appeared by an ultrasonic treatment, using a hydrogen-containing irrigating solution.

FIG. 15 includes photographs of the anterior segments of both eyes (A1 and B-1) and photographs of corneal endothelial cells (A-2 and B-2), before the surgery.

FIG. 16 includes photographs of anterior segments (A-1 and B-1) and photographs of corneal endothelial cells (A-2 and B-2), on Day 1 after the surgery.

FIG. 17 includes photographs of anterior segments (A-1 and B-1) and photographs of corneal endothelial cells (A-2 and B-2), on the 3rd week after the surgery.

FIG. 18 is a view showing the results obtained by mapping the cornea thickness using an anterior eye part analyzer on the 3rd week after the surgery.

### DESCRIPTION OF EMBODIMENTS

Hereinafter, the present invention will be described in detail.

The prophylactic or therapeutic agent for oxidative damage for use in intraocular surgery of the present invention is a liquid composition comprising at least hydrogen molecules. Hydrogen molecules can be dissolved in water or an aqueous solution for a certain period of time. Such water or an aqueous solution, in which hydrogen molecules are present in a saturated state, can be easily produced by dissolving hydrogen gas in water or an aqueous solution in a pressurized state, and then removing the pressure. For example, an aqueous solution may be left under the pressure of hydrogen gas at 0.4 MPa or more, for several hours, and preferably for 1 to 3 hours. Alternatively, an aqueous solution may be placed in a vessel filled with hydrogen gas for several days, and preferably for approximately 1 day. Otherwise, such water or an aqueous solution may also be produced in a short time, using an apparatus for producing a large amount of hydrogen water. Such an apparatus may be, for example, an apparatus, which allows pressurized hydrogen gas to directly come into contact with a pressurized liquid that flows through a pipeline thereof, so that hydrogen molecules can be efficiently and promptly dissolved in the liquid.

17.5 ml of hydrogen molecules can be dissolved in 1 L of water at 25° C. at 1 atm (approximately 0.8 mM). The prophylactic or therapeutic agent for oxidative damage for use in intraocular surgery of the present invention, which is a liquid composition comprising hydrogen molecules, comprises hydrogen molecules at a concentration of 0.1 mM or higher, preferably 0.4 mM or higher, and particularly preferably 0.5 mM or higher.

The prophylactic or therapeutic agent for oxidative damage for use in intraocular surgery of the present invention is preferably used as an intraocular irrigating solution.

The prophylactic or therapeutic agent for oxidative damage for use in intraocular surgery of the present invention can be produced by dissolving hydrogen in an intraocular irrigating solution comprising potassium chloride, magnesium chloride, calcium chloride hydrate, sodium chloride, dibasic sodium phosphate hydrate, sodium hydrogen carbonate, sodium acetate hydrate, sodium citrate hydrate, sodium hydroxide, hydrochloric acid, glucose, etc. The prophylactic or therapeutic agent for oxidative damage for use in intraocular surgery of the present invention can further comprise additives exhibiting a barrier function-protecting action or a pump function-protecting action on corneal endothelium, which are used in common intraocular irrigating solutions. As such an additive, oxyglutathione is used for example. The pH value of the prophylactic or therapeutic agent for oxidative damage for use in intraocular surgery of the present invention may be in a range that is acceptable as an intraocular irrigating solution, and it is preferably in the range of approximately pH 7 to 8.

The prophylactic or therapeutic agent for oxidative damage for use in intraocular surgery of the present invention may be administered in the form of an intraocular irrigating solution, when prophylaxis or treatment is carried out by intraocular surgery such as phacoemulsification cataract surgery. The present prophylactic or therapeutic agent for oxidative damage can be used in intraocular surgery in which an intraocular irrigating solution is used. Examples of such surgery include surgeries for cataract, vitreous body, and glaucoma. Examples of the cataract surgery include intracapsular cataract extraction, extracapsular cataract extraction (posterior chamber intraocular lens implantation), and phacoemulsification and aspiration. Examples of the vitreous surgery include diabetic retinopathy surgery and retinal detachment surgery. In addition, examples of the glaucoma surgery include iridectomy, trabeculectomy, and trabeculoplasty.

The amount of the prophylactic or therapeutic agent for oxidative damage for use in intraocular surgery of the present invention, which is used in a single intraocular surgery, is not particularly limited. The used amount is appropriately increased or decreased depending on a surgical form, a surgical time and the like. The rough guide therefor is as follows.

| | | |
|---|---|---|
| Cataract surgery | 20 to 500 | mL |
| Vitreous surgery | 50 to 4,000 | mL |
| Glaucoma surgery | 20 to 50 | mL |

In phacoemulsification cataract surgery, the present prophylactic or therapeutic agent for oxidative damage can be used in combination with an ophthalmic viscoelastic agent consisting of a viscoelastic material such as sodium hyaluronate or sodium chondroitin sulfate, which is used for the purpose of facilitating surgery involving the maintenance of the form of the cornea and protecting corneal endothelial cells. An ophthalmic viscoelastic agent is injected into an eye, and an anterior capsule of lens is then circumferentially excised. At this time, while the prophylactic or therapeutic agent for oxidative damage for use in intraocular surgery of the present invention is injected as an irrigating solution into a crystalline lens, the nucleus of the crystalline lens is disintegrated by ultrasound, and the emulsified nucleus and cortex are then aspirated. Thereafter, an intraocular lens is inserted into the excised portion, and the prophylactic or therapeutic agent for oxidative damage for use in intraocular surgery is then used, so that the intraocular pressure can be returned to a normal pressure. Thereby, oxidative damage generated as a result of the ultrasound can be suppressed, and transition to bullous keratopathy can also be suppressed.

The effects obtained by using the prophylactic or therapeutic agent for oxidative damage for use in intraocular surgery of the present invention as an irrigating solution can be confirmed by observing the presence or absence of corneal opacity in gross appearance or under a microscope. By using the prophylactic or therapeutic agent for oxidative damage for use in intraocular surgery of the present invention, corneal opacity is significantly suppressed.

Otherwise, the expression of heme oxygenase-1 (HO-1) that is a factor induced by oxidative stress may be confirmed in corneal endothelial cells. The expression of HO-1 can be confirmed by measuring mRNA or by measuring a protein. By using the prophylactic or therapeutic agent for oxidative damage for use in intraocular surgery of the present invention, the expression of HO-1 is significantly suppressed in corneal endothelial cells.

Alternatively, as oxidative stress markers in corneal endothelial cells, 4-hydroxynonenal (4-HNE) that indicates lipid peroxidation and 8-hydroxy-2-deoxyguanosine (8-OHdG) that indicates the oxidation of a nucleic acid may also be measured. By using the prophylactic or therapeutic agent for oxidative damage for use in intraocular surgery of the present invention, the number of cells positive to 4-HNE or 8-OHdG is significantly decreased.

Hereinafter, the present invention will be specifically described in the following Examples. However, these examples are not intended to limit the scope of the present invention.

In the following Examples, corneal disorder models were produced from domestic rabbits according to ultrasound oscillation in the anterior chamber, and upon the production of the disorder models, a prophylactic or therapeutic agent for oxidative damage for use in intraocular surgery was perfused, so that the effects of the present invention were confirmed. Five hours after the production of the disorder models, individual analyses were carried out. After the approval of the Animal Care and User Committee the Nippon Medical School, had been obtained, the animals were handled in accordance with the ARVO Statement for the Use of Animals in Ophthalmic and Vision Research.

In the Examples conducted in clinical sites, phacoemulsification and aspiration were carried out on both eyes of patients with serious cataract, and thereafter, a hydrogen-containing intraocular irrigating solution was used for only one eye, so that the effects of the present invention were confirmed. After the approval of the ethics committee of Nippon Medical School had been obtained, clinical studies were carried out in accordance with the “ethical guidelines for clinical research” of the Japanese Ministry of Health, Labour and Welfare.

### Example 11

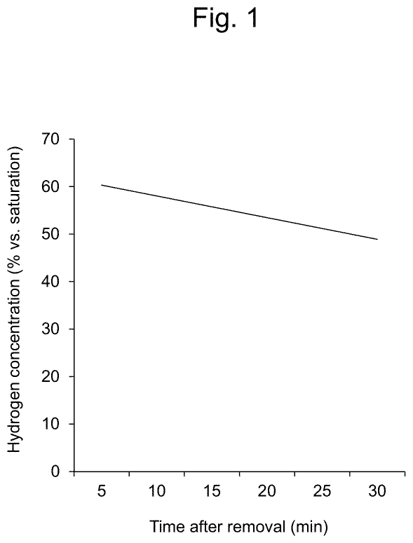

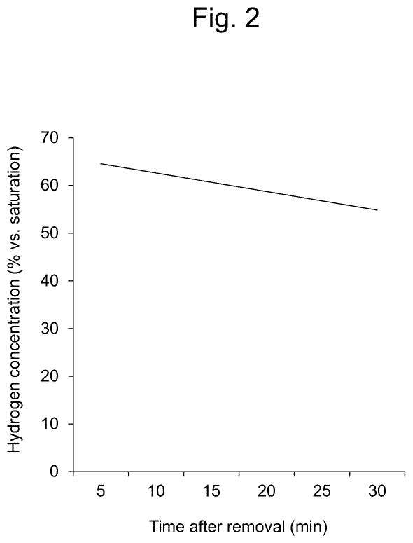

A prophylactic or therapeutic agent for oxidative damage for use in intraocular surgery was prepared by the following method. One package (500 mL) of intraocular irrigating solution, OPEGUARD NEO KIT OCULAR IRRIGATING SOLUTION 0.0184% in a soft plastic bag (Senju Pharmaceutical Co., Ltd.), was indwelled in an acrylic box (inner dimension: 260 W×260 D×100 H; acrylic vacuum desiccator SNS type; manufactured by SANPLATEC CO., LTD.) filled with 100% hydrogen gas, for 24 hours or 1 week. Subsequently, a change in the hydrogen concentration in the intraocular irrigating solution, which had been removed from the acrylic box, was measured. For the measurement of the hydrogen concentration, a needle-type hydrogen sensor (H2-N; manufactured by Unisense), which had been subjected to two-point correction with pure water and a hydrogen saturated water thereof immediately before the measurement, was used. In the case of indwelling for 24 hours, the hydrogen concentration was found to be 0.49 mM (60% of saturation) at the time of opening. Then, at 30 minutes after the opening, the hydrogen concentration was found to be 0.41 mM (50% of saturation) (FIG. 1). On the other hand, in the case of indwelling for 1 week, the hydrogen concentration was found to be 0.53 mM (65% of saturation) at the time of opening. Then, at 30 minutes after the opening, the hydrogen concentration was found to be 0.45 mM (55% of saturation) (FIG. 2). In the present example, the prophylactic or therapeutic agent for oxidative damage for use in intraocular surgery, which has been indwelled in a box for 24 hours, and after the opening of the box, has been left for 30 minutes, is referred to as a “hydrogen-containing irrigating solution” for convenience sake, and an irrigating solution before addition of hydrogen is referred to as a “normal irrigating solution.” These irrigating solutions were used in the following examples.

### Example 2

Domestic rabbits (body weight: 2.5 kg to 3.0 kg; Japanese white rabbits; Tokyo Laboratory Animals Science Co. Ltd.), which had been anesthetized using ketamine (30 mg/kg body weight; trade name: Ketalar; DAIICHI SANKYO COMPANY, LIMITED.) and xylazine (4 mg/kg body weight; trade name: Celactal; Bayer Yakuhin, Ltd.) according to a common method, were laid on their side. To the eyeball, a tropicamide-phenylephrine ophthalmic solution (trade name: Mydrin-P Ophthalmic Solution; Santen Pharmaceutical Co., Ltd.) was added dropwise as a mydriatic agent, and also, an oxybuprocaine hydrochloride solution (trade name: Benoxil Ophthalmic Solution; Santen Pharmaceutical Co., Ltd.) was added dropwise as a topical anesthesia. Corneal excision was carried out using an ophthalmic slit knife, and an ultrasound probe connected with a cataract surgical device (Stellaris (registered trademark); Bausch & Lomb) was inserted into the anterior chamber. The cataract surgical device was set under conditions equivalent to those in human cataract surgery (i.e., ultrasound output: 30%, continuous oscillation: 90 seconds, aspiration pressure: 185 mmHg, irrigating solution bottle height: 75 cm, irrigating solution flow rate: 25 mL/min), and ultrasonic oscillation was then carried out. After completion of the oscillation, the ultrasound probe was moved and each domestic rabbit was then left at rest.

### Example 3

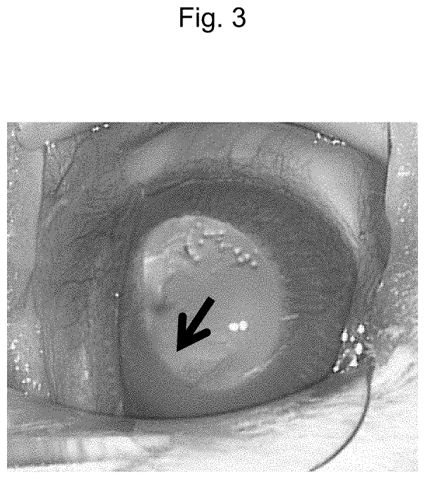

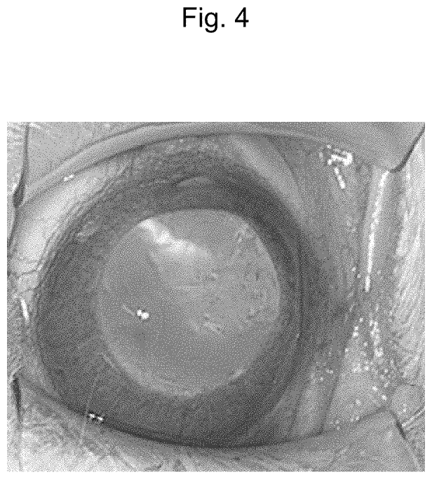

After the ultrasonic oscillation had been carried out for 5 hours, the anterior segment was observed under an ophthalmologic surgical microscope. In the case of using a normal irrigating solution, it was confirmed that opacity was generated by corneal edema (FIG. 3; arrow). On the other hand, in the case of using a hydrogen-containing irrigating solution, corneal opacity was not observed in almost all cases (FIG. 4).

### Example 4

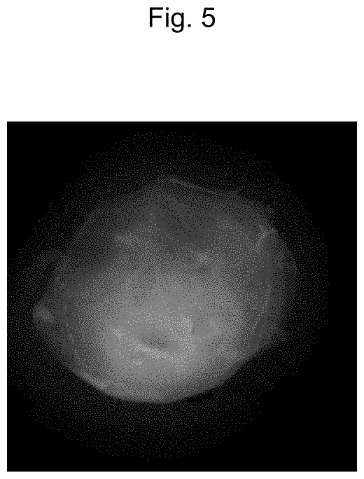

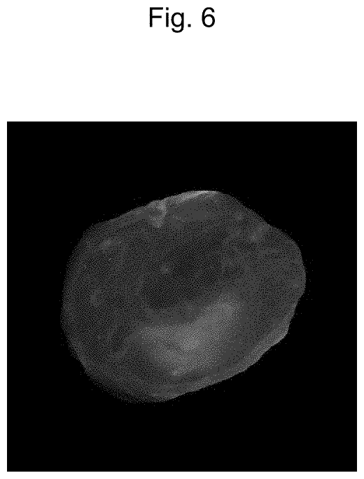

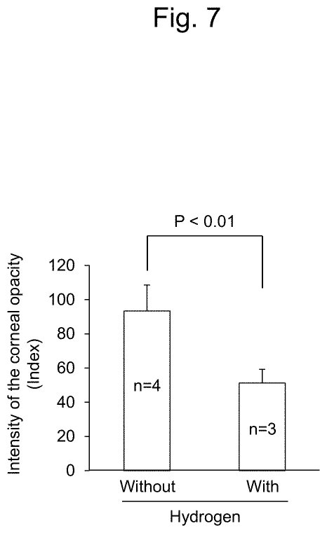

After the ultrasonic oscillation had been carried out for 5 hours, the cornea was excised into a round shape, and a digital image (240 mm in width×180 mm in height, 640 pixels in width×480 pixels in height) was then photographed under a stereoscopic microscope. In comparison to the cornea to which the normal irrigating solution was used (FIG. 5), in the cornea to which the hydrogen-containing irrigating solution was used (FIG. 6), opacity caused by ultrasound was reduced. In order to evaluate corneal opacity, opacity (clouded portion) in the central portion of the cornea (500 pixels×500 pixels, 250,000 pixels in total) was quantified using the image analysis software ImageJ (NIH). As a result, it was found that opacity was significantly reduced in the cornea to which the hydrogen-containing irrigating solution was used (FIG. 7).

### Example 51

Heme oxygenase-1 (HO-1) is a factor induced by oxidative stress. In the upstream thereof, the transcriptional factor Nrf2 and the hypoxia-inducible factor HIF1 are present. As such, the expression of HO-1 can be used as a marker for measuring the degree of oxidative stress. Hence, the expression of HO-1 in corneal endothelial cells was examined at a transcriptional level of mRNA. For the measurement, a quantitative PCR method of using SYBR Green was applied. Specifically, total RNA was extracted from cornea that had been isolated after the ultrasonic oscillation for 5 hours, using an RNA extraction kit (Qiagen). The obtained total RNA was then subjected to reverse transcription using an RT-PCR kit (Takara Bio, Inc.) to obtain complementary DNA. Subsequently, using Takara Ex Taq (Takara Bio, Inc.) and a pair of DNA primers as shown below, quantitative PCR was carried out. It is to be noted that the mRNA of glyceraldehyde-3-phosphate dehydrogenase (GAPDH) was simultaneously quantified, and was then used as an internal standard.

The following pair of DNA primers were used, respectively.

HO-1
(SEQ ID NO: 1)
5′-CAGGTGACTGCCGAGGGTTTTA-3′ (forward)
(SEQ ID NO: 2)
5′-GGAAGTAGAGCGGGGCGTAG-3′ (reverse)
GAPDH
(SEQ ID NO: 3)
5′-GCCGCTTCTTCTCGTGCAG-3′ (forward)
(SEQ ID NO: 4)
5′-ATGGATCATTGATGGCGACAACAT-3′ (reverse)

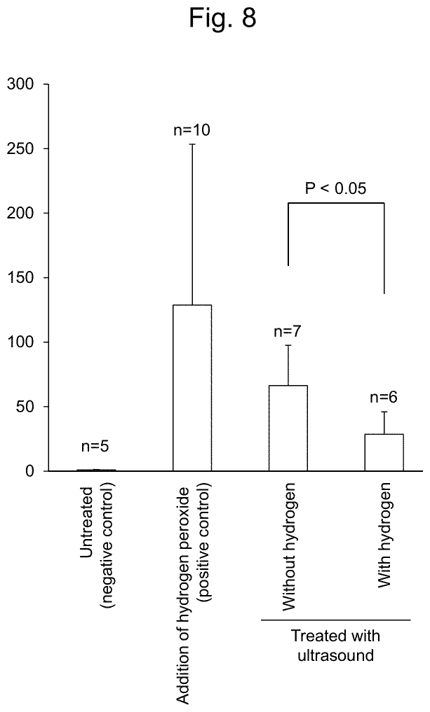

As a PCR device, 7500 Fast Real-Time PCR System (manufactured by Life Technologies, Inc.) was used, and a PCR cycle consisting of 90° C.-10 seconds, 95° C.-5 seconds, and 60° C.-34 seconds was carried out 40 times. Consequently, it was found that although reactive oxygen species generated as a result of the ultrasound induced HO-1, the expression of the HO-1 was significantly decreased as a result of a decrease in the reactive oxygen species due to a hydrogen-containing irrigating solution (FIG. 8).

### Example 6

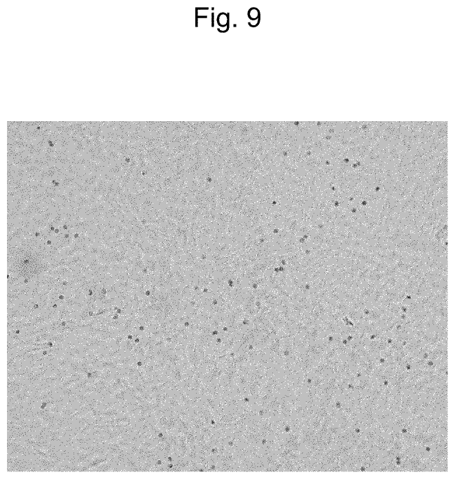

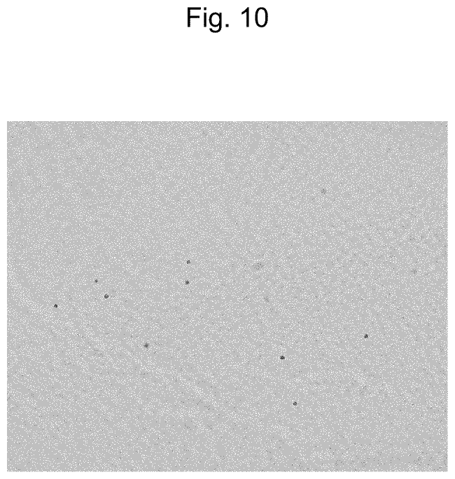

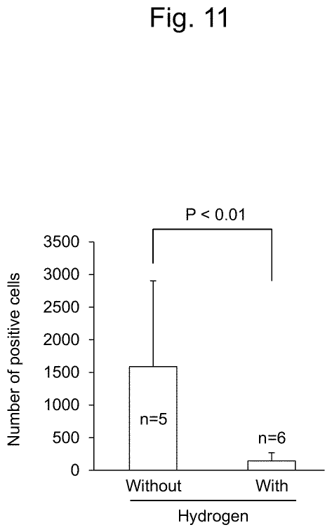

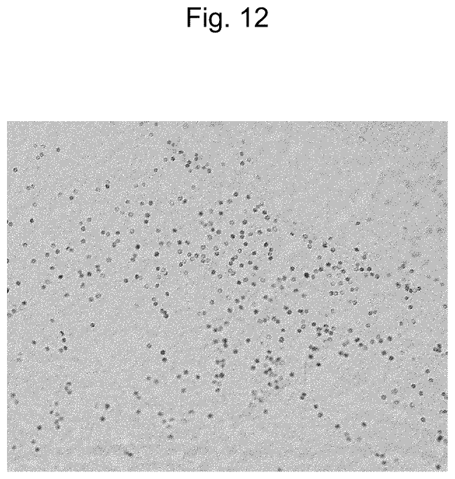

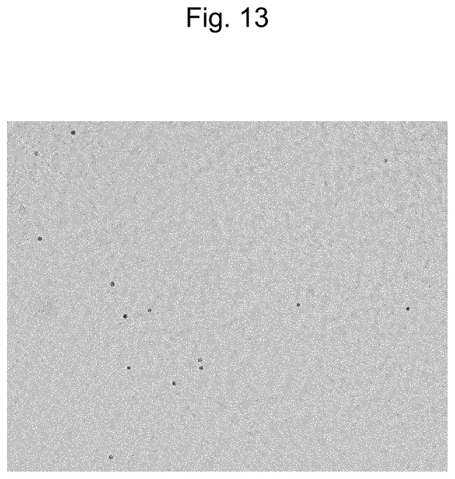

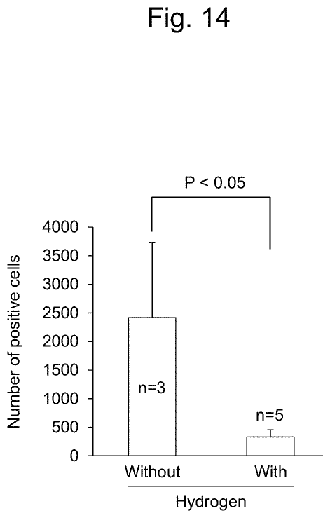

As oxidative stress markers in corneal endothelial cells, 4-HNE that indicates lipid peroxidation and 8-OHdG that indicates the oxidation of a nucleic acid were examined Detection was carried out by staining with anti-4-HNE and 8-OHdG antibodies. First, the excised corneas was fixed with a Bouin's solution for 1 hour to produce a flat mount of cornea having endothelial cells thereof as an upper surface. For immunostaining, VECTASTAIN ABC System (Vector Laboratories) was employed, and a 1:30 dilution of anti-4-HNE and anti-8-OHdG antibodies were used as a primary antibody. Both antibodies were obtained from Japan Institute for the Control of Aging. As a secondary antibody, a 1:66 dilution of biotin-labeled anti-mouse IgG antibody was used. For visualization, an avidin-biotin-labeled peroxidase complex and its substrate 3,3′-diaminobenzidine (DAB) were used. As a result of the ultrasound, the number of 4-HNE-positive cells was increased in the corneal endothelium (FIG. 9). However, the number was drastically decreased with the use of a hydrogen-containing irrigating solution (FIG. 10). The number of 4-HNE-positive cells was counted, and as a result, a significant decrease in the number was found (FIG. 11). Moreover, as a result of the ultrasound, the number of 8-OHdG-positive cells was increased in the corneal endothelium (FIG. 12). However, the number was drastically decreased with the use of a hydrogen-containing irrigating solution (FIG. 13). The number of 8-OHdG-positive cells was counted, and as a result, a significant decrease in the number was found (FIG. 14).

From the aforementioned experiment, it could be predicted that, in order to suppress oxidative damage in intraocular surgeries including phacoemulsification cataract surgery, the inside of an eye is perfused with a hydrogen molecule-containing prophylactic or therapeutic agent for oxidative damage generated in intraocular surgeries, so that good prognosis can be maintained.

### Example 7

Phacoemulsification and aspiration were carried out on both eyes of a 66-year-old female patient with serious cataract of both eyes, who had Emery classification of 4.5 and a preoperative vision of 0.01.

The details of the patient were as follows.

### Patient:

66 years old, female, cataract of both eyes (Emery classification: 4.5 (both eyes)) Intraocular irrigating solution used:
Right eye: Hydrogen-containing intraocular irrigating solution (prepared by the method described in Example 1)
Left eye: Normal intraocular irrigating solution (OPEGUARD NEO KIT OCULAR IRRIGATING SOLUTION 0.0184%, Senju Pharmaceutical Co., Ltd.)

### Conditions for Ultrasonic Oscillation:

Apparatus used: Stellaris, B. L. J. Company, Ltd.; conditions: right eye: Ave 27%, APT 70.15 seconds, EPT 18.94 seconds, left eye: Ave 25%, APT 67.1 seconds, EPT 16.78 seconds (Ave: average ultrasound output, APT: actual ultrasonic oscillation time, EPT: equivalent ultrasonic oscillation time)

### Vision Change:

Before surgery: both eyes 20/2000 (0.01); Day 1 after surgery: right eye 20/25 (0.8), left eye 20/50 (0.4), 1 week after surgery: both eyes 20/22 (0.9); 3 weeks after surgery: right eye 20/20 (1.0), left eye 20/25 (0.8)
Change in number of corneal endothelial cells (cells/mm²):
Before surgery: right eye 2890, left eye 2994; Day 1 after surgery: right eye 2833, left eye 613; 1 week after surgery: right eye 2899, left eye 1106; 3 weeks after surgery: right eye 2907, left eye 1186

The hydrogen-containing intraocular irrigating solution of the present invention was used for the right eye, whereas a normal intraocular irrigating solution containing no hydrogen was used for the left eye. The ultrasound was applied to both eyes, so that the crystalline lens was emulsified.

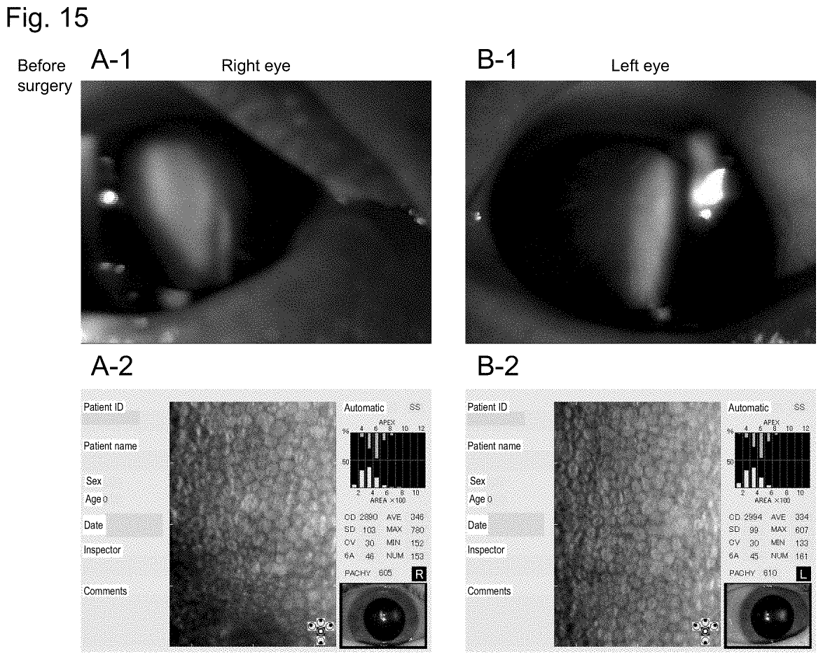

FIG. 15 includes photographs of the anterior segments of both eyes (A1 and B-1) and photographs of corneal endothelial cells (A-2 and B-2), before the surgery. Almost no difference was found between the corneal endothelial cells of both eyes.

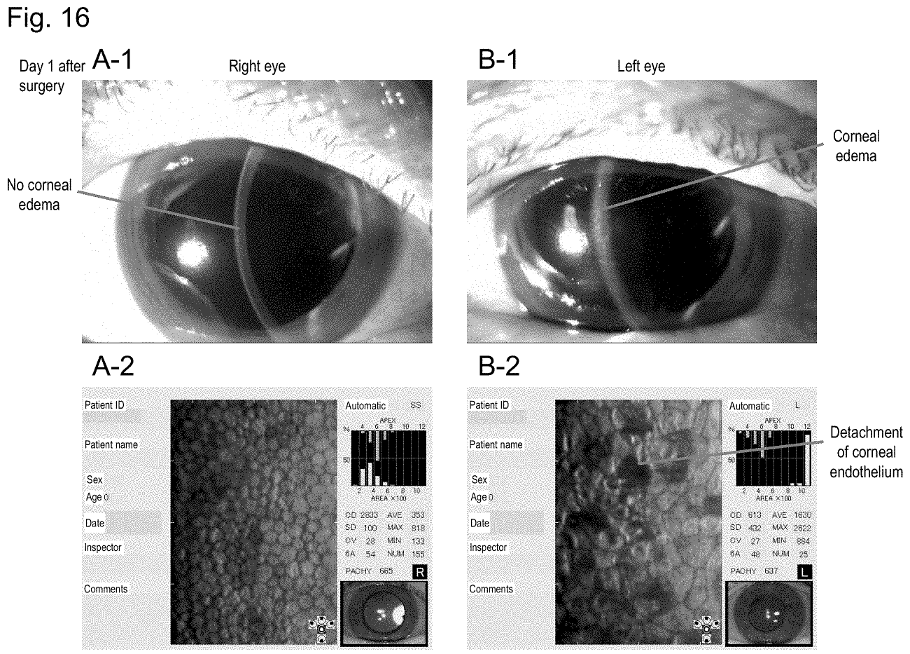

FIG. 16 includes photographs of anterior segments (A-1 and B-1) and photographs of corneal endothelial cells (A-2 and B-2), on Day 1 after the surgery. In the left eye for which the normal intraocular irrigating solution was used, corneal edema (B-1) and the detachment of corneal endothelial cells (B-2) were found. In contrast, in the right eye for which the hydrogen-containing intraocular irrigating solution was used, there were no such findings (A-1 and A-2).

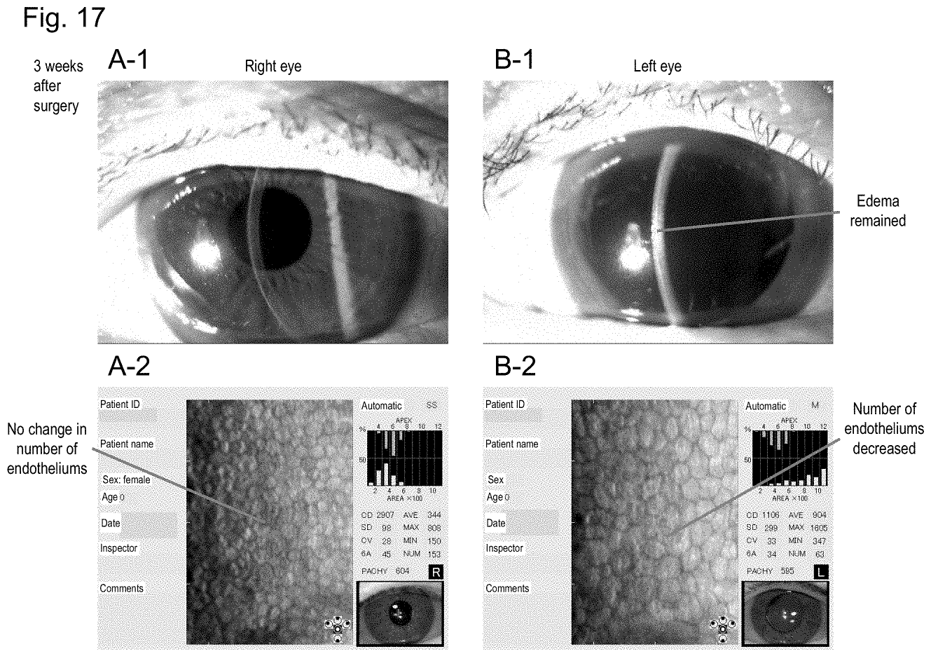

FIG. 17 includes photographs of anterior segments and photographs of corneal endothelial cells, on the 3rd week after the surgery. In the left eye for which the normal intraocular irrigating solution was used, corneal edema remained (B-1), and the number of corneal endothelial cells was significantly decreased (B-2). In contrast, in the right eye for which the hydrogen-containing intraocular irrigating solution was used, there were no such findings (A-1 and A-2).

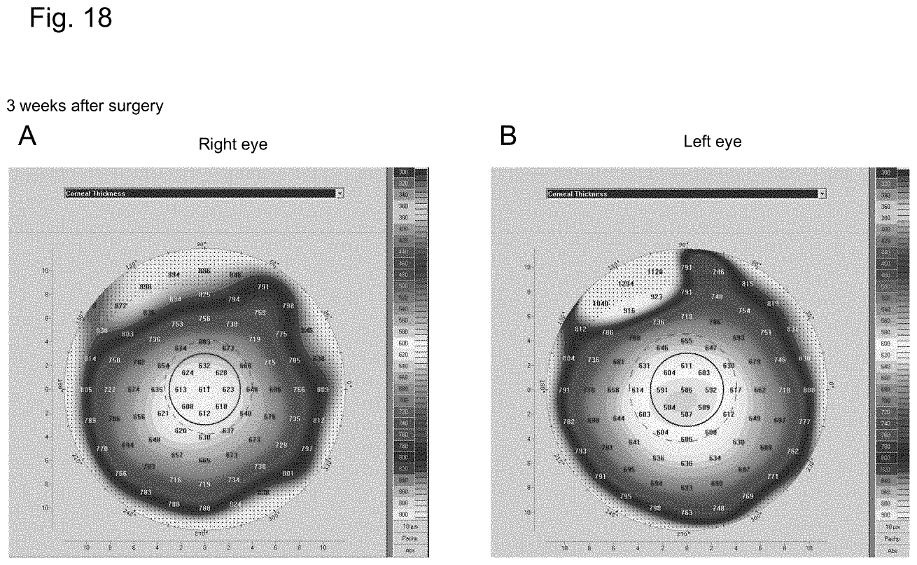

FIG. 18 is a view showing the results obtained by mapping the cornea thickness using an anterior eye segment tomography (Pentacam, Oculus, Germany) on the 3rd week after the surgery. In the right eye for which the normal intraocular irrigating solution was used, the thickness of the cornea in the direction of 11 o'clock was increased due to corneal edema (B; light blue portion (which is a white color in the black and white photograph)).

Although the conditions for ultrasonic oscillation were almost equivalent in both eyes, with regard to the vision after the surgery, the right eye, for which the hydrogen-containing intraocular irrigating solution was used, exhibited a high value, and a decrease in the number of corneal endothelium and corneal edema, which were found in the left eye, were not found in the right eye.

Therefore, the right eye subjected to the surgery using the hydrogen-containing intraocular irrigating solution had much better prognosis than the left eye subjected to the surgery using the normal intraocular irrigating solution. These results show the effectiveness of the hydrogen-containing intraocular irrigating solution.

### INDUSTRIAL APPLICABILITY

The hydrogen molecule-containing prophylactic or therapeutic agent for oxidative damage generated in intraocular surgeries of the present invention is useful as a prophylactic or therapeutic agent for oxidative damage generated in intraocular surgeries, which is administered to suppress oxidative damage generated in the intraocular surgeries.

All publications, patents and patent applications cited in the present description are incorporated herein by reference in their entirety.

1. A prophylactic or therapeutic agent for oxidative damage, comprising hydrogen molecules, wherein the prophylactic or therapeutic agent is for use in intraocular surgery.

2. The prophylactic or therapeutic agent for oxidative damage for use in intraocular surgery according to claim 1, wherein the prophylactic or therapeutic agent comprises hydrogen molecules at a concentration of 0.4 mM or higher.

3. The prophylactic or therapeutic agent for oxidative damage for use in intraocular surgery according to claim 1, wherein the prophylactic or therapeutic agent is an irrigating solution.

4. The prophylactic or therapeutic agent for oxidative damage for use in intraocular surgery according to claim 1, which is administered during phacoemulsification cataract surgery.

5. The prophylactic or therapeutic agent for oxidative damage for use in intraocular surgery according to claim 4, which is continuously administered to eyes via perfusion administration during intraocular surgery.

6. The prophylactic or therapeutic agent for oxidative damage for use in intraocular surgery according to claim 4, which is used in combination with injection of a viscoelastic material.

7. A method of treating or preventing oxidative damage during ocular surgery, comprising administering to an eye of a patient in need thereof a composition comprising hydrogen molecules.

8. The method of claim 7, wherein the composition is applied to the eye as an irrigating solution.

9. The method of claim 7, wherein the composition is applied during phacoemulsification cataract surgery.

10. The method of claim 7, wherein the intraocular surgery is selected from the group consisting of cataract surgery, vitreous surgery and glaucoma surgery.

11. The method of claim 7, wherein the composition is administered in combination with a viscoelastic material.

12. The method of claim 7, wherein the composition comprises hydrogen molecules at a concentration of 0.4 mM or higher.


SEQUENCE LISTING (4 sequences)

SEQ ID NO: 1 - DNA, 22 bp, Artificial
  Primer
caggtgactgccgagggtttta

SEQ ID NO: 2 - DNA, 20 bp, Artificial
  Primer
ggaagtagagcggggcgtag

SEQ ID NO: 3 - DNA, 19 bp, Artificial
  Primer
gccgcttcttctcgtgcag

SEQ ID NO: 4 - DNA, 24 bp, Artificial
  Primer
atggatcattgatggcgacaacat